(19) United States
(12) Patent Application Publication
(10) Pub. No.: US 2021/0169388 A1
(43) Pub. Date: Jun. 10, 2021

(54) METHOD AND APPARATUS FOR DETERMINING HEMISPHERIC EMOTIONAL VALENCE

(71) Applicant: Mindlight, LLC, Newton, MA (US)

(72) Inventor: Frederic Schiffer, Newton, MA (US)

(21) Appl. No.: 16/703,937

(22) Filed: Dec. 5, 2019

(51) Int. Cl. A61B 5/16; A61B 5/00

(52) U.S. Cl. CPC A61B 5/165; A61B 5/4082; A61B 5/4845; A61B 5/163; A61B 5/4884; A61B 5/4088

(57) ABSTRACT

An apparatus and method for determining the hemispheric emotional valance of a person is disclosed. The apparatus assists in the selection of appropriate treatment modalities for various neurological disorders. A computer outputs a series of preselected stimuli and a screen is adapted to display the preselected stimuli to the person. The stimuli may comprise a focal point presented on the screen for the person to fixate on and a picture. The focal point is positioned on the screen so that the images will be seen in the person's lateral visual field. The focal point and the picture are displayed to the person for a preselected period of time to induce stress or comfort as the mental state associated with one of the cerebral hemispheres by stimulating the contralateral hemisphere. The level of reaction to the stimuli is recorded in the computer or manually.

### FIELD OF THE INVENTION

This invention relates generally to the field of psychiatry and neurology and more particularly to a method and apparatus for determining which hemisphere of the brain has a more positive valence so that appropriate treatment modalities may be applied thereto.

### BACKGROUND OF THE INVENTION

It is well known that the application of intense light, a non-ionizing phototherapy, has been reported in over a thousand scientific publications to have therapeutic efficacy for a wide range of disorders in humans without any observed harmful effects. Light has been demonstrated in cell culture to increase mitochondrial respiration, increase ATP synthesis, increase heat shock proteins, induce transforming growth factor β-1, and increase nerve cell proliferation and migration. Light has been tested in animals and found to facilitate wound healing, promote the process of skeletal muscle regeneration, and reduce infarct size in ischemic heart muscle by 50% to 70% in an induced experimental model in rats and dogs. Light in the near infrared spectrum, which penetrates the scalp and skull, can significantly reduce damage from experimentally induced stroke in rats and rabbits, and to improve memory performance of middle-aged mice. It has also been shown in humans to improve symptoms of traumatic brain injury and of Alzheimer's disease.

My prior work as disclosed in U.S. Pat. Nos. 8,303,636 and 8,574,279 teach methods for treating psychiatric disorders using light energy, the subject matter of which is incorporated herein. In addition to providing a method of treating the brain using near infrared light (NIR), the patents also teach a method of diagnosing which hemisphere requires treatment. In accordance with treatment standards each patient had a Standard Clinical Diagnostic Interview to determine their diagnosis. The patients also had their hemispheric emotional valence (HEV) measured using lateral visual field stimulation (LVFS), in which the patient was instructed to look out of one side of taped safety glasses to allow their vision to be restricted to either the left or the right lateral visual field. The glasses were taped or covered to permit vision to only one lateral visual field at a time. The patient was asked to look to one side and to fixate the center of his vision on the edge of the tape so that he or she was looking out of the lateral half of one eye. Vision from the other eye was occluded by tape on the opposite side. While looking out of the specific visual field, the patient was asked to look at a photograph of a man or woman with an angry facial expression. After 45 seconds, he or she was asked to verbally rate his or her feelings for each of 10 affects from Positive and Negative Affect Scale (PANAS), from none to extreme on a 5-point scale. Following the PANAS measurements, the patient was then asked to repeat the procedure by looking out of the second visual field.

The PANAS has 5 positive affects including alert, inspired, determined, attentive and active and 5 negative affects including upset, hostile, ashamed, nervous and afraid. The score measured when the person looks out of the right lateral visual field (RVF) can be subtracted from the score measured when the person looks out of the left lateral visual field (LVF). The LVF is indicative of the state of the right hemisphere since the connection between the medial retinas and the cerebral hemispheres are crossed. Since a more positive PANAS score indicates more positive affect, we assign a value for the person's

HEV according to the formula: LVF PANAS score—RVF PANAS. A positive score suggests that the right hemisphere has a more positive HEV. Research studies have shown that LVFS offers a good indication that the left and right cerebral hemispheres are generally associated with different emotional valences such the one hemisphere (either left or right) is associated with a more positive outlook on the world (positive hemispheric valence) and the other a more negative outlook on the world (negative hemispheric valence) and HEV has also been shown to correlate highly with significant brain anatomy.

While the foregoing test could be used to determine the HEV of a patient, it was not without its drawbacks and deficiencies. For example, the patient had to put on clumsy safety goggles which were uncomfortable and difficult to put on for some patients. In addition, researchers and clinicians found the taped goggles to appear unprofessional, and often felt that the patient could be easily biased by the treating clinician or researcher.

In view of the foregoing it is an object of the present invention to provide an improved method and apparatus for determining which side of the brain has a higher emotional valence.

Another object of the present invention is to provide an improved method and apparatus for determining which side of the brain has a higher emotional valence that is more objective and employs a more scientific method.

Yet another object of the present invention is to provide an improved method and apparatus for determining which side of the brain has a higher emotional valence that is easier to use.

Yet another object of the present invention is to provide an improved method and apparatus for determining which side of the brain has a higher emotional valence that can be self administered.

Still another object of the present invention is to provide an improved method and apparatus for determining which side of the brain has a higher emotional valence that is less intimidating to the patient.

A still further object of the present invention is to provide an improved method and apparatus for determining which side of the brain has a higher emotional valence that is less susceptible to clinician and/or patient bias.

Another object of the present invention is to provide a method and apparatus that can be used to determine a person's HEV over the World Wide Web/internet.

Another object of the present invention is to provide a method and apparatus that can be used to measure the outcome of a medical intervention or treatment.

### SUMMARY OF THE INVENTION

To accomplish the objects described above there is provided an apparatus and method for determining the hemispheric emotional valance of a person. The apparatus assists in the selection of appropriate treatment modalities for cognitive enhancement, emotional well-being, psychiatric disorders including anxiety, depression, PTSD, drug abuse; neurological disorders including Alzheimer's disease, Parkinson's disease and traumatic brain injury. The apparatus comprises a computer adapted to output a series of preselected stimuli and a screen adapted to display the preselected stimuli to the person. The stimuli may comprise a focal point presented on the screen for the person to fixate on and a picture. The focal point is positioned on the screen so that the images will be seen in the person's lateral visual field. The focal point and the picture are displayed to the person for a preselected period of time to induce stress or comfort as the mental state associated with one of the cerebral hemispheres by stimulating the contralateral hemisphere. The level of reaction to the stimuli is recorded in the computer or manually.

### BRIEF DESCRIPTION OF THE DRAWINGS

FIG. 1 is a perspective view of a person self-administering the HEV test on a lap top computer.

FIG. 2 is a view of a first test image appearing on the laptop screen.

FIG. 3 is a view of a second test image appearing on the laptop screen.

### DETAILED DESCRIPTION OF THE ILLUSTRATED EMBODIMENT

While the present invention will be described more fully hereinafter with reference to the accompanying drawings, in which a particular embodiment is shown, it is to be understood at the outset that persons skilled in the art may modify the invention herein described while still achieving the favorable results of this invention. Accordingly, the description which follows is to be understood as a broad teaching disclosure directed to persons of skill in the appropriate arts and not as limiting upon the present invention.

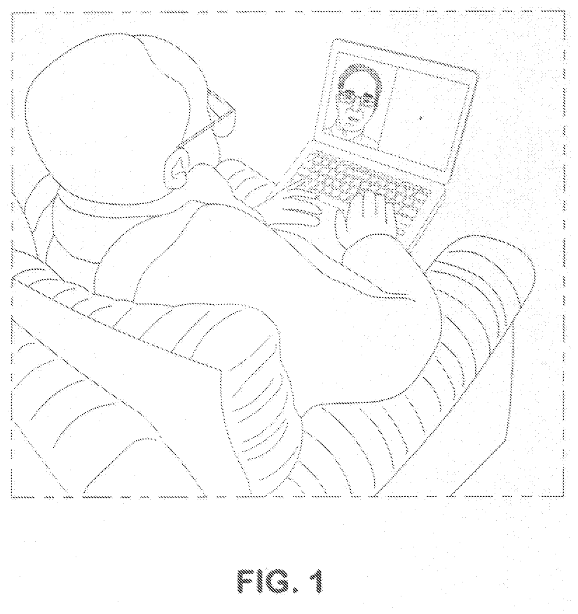

Referring now specifically to the drawings and specifically to FIG. 1, the apparatus for determining the hemispheric emotional valance of a person to assist in the selection of appropriate treatment modalities for determining which side of the brain exhibits dominant HEV is resident on a lap top computer. The laptop computer is positioned on the subject's lap so that the screen is visible and the keypad is useable for data entry. It will be noted that while FIG. 1 shows a subject using a laptop, this is for illustrative purposes only and that the computer may be variously positioned such as on a tabletop or desktop. Additionally, any other type of computer or display device (such as CRT or LED screens) may be employed with equal efficacy. Computers, whether they be laptop, desktop or cloud based, comprise a number of components which may be variously connected depending on type. It is well known that the laptop computer comprises a keyboard, a display device such as an LED screen, a mouse type device for moving the cursor about the screen and an internally positioned CPU (central processing unit—not shown) that computes and controls data flow, analysis and storage between the constituent parts and memory. All of the foregoing components and the signals flow there between.

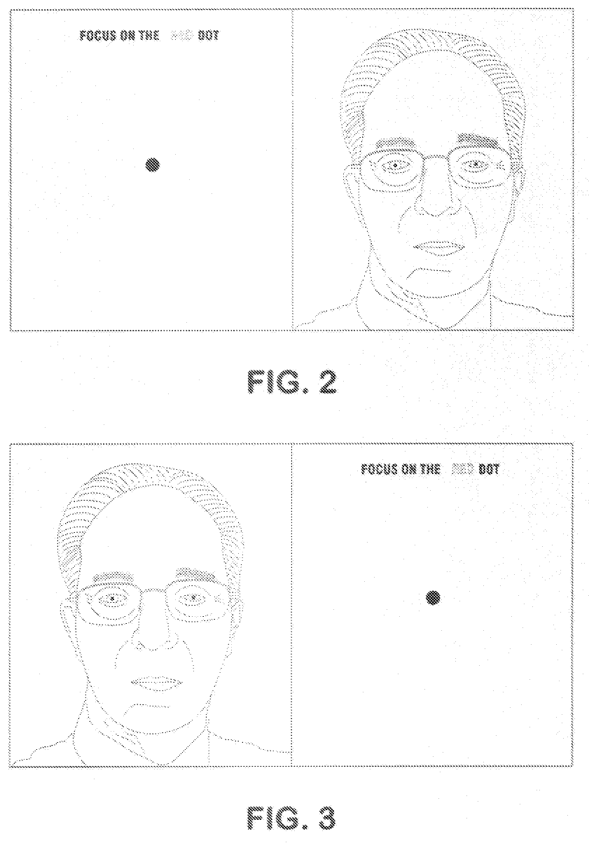

The computer is adapted to output a series of preselected stimuli to the screen generally in any form that will stimulate one side of the brain or the other to determine the person's HEV. The stimuli in the illustrated embodiment as shown in FIGS. 2 and 3 comprise a focal point presented on the screen for the person to fixate on. As employed herein the term “focal point” may also be described as a “point of focus”. The focal point is positioned on the screen spaced apart from an image or picture. The image, such as a photograph is positioned on the extreme side of the screen so that it is seen in the person's lateral visual field when the person fixates on the focal point. It will be noted that the photograph is quite large, but no particular dimension is required so long as it is positioned in the lateral visual field when the person fixates on the focal point (dot). Likewise, the fixation dot is centrally located on the opposite side of the screen. No particular color, size or number is required so long as the person fixates thereon and the photograph is presented to the person's lateral visual field. The focal point or points should be positioned in horizontal alignment along the horizontal centerline of the screen. When multiple dots are employed, they should be spaced apart from each other and may be unique (for example, different colors), thus allowing the person to choose which one to fixate on. The photograph may display an upsetting (angry), neutral or pleasant (smiling) face depending upon what emotion is intended to be evoked. The focal point and picture are displayed to the person for a preselected period of time as stated above and induces stress or comfort as the mental state associated with one of the cerebral hemispheres by stimulating the contralateral hemisphere and the level of reaction to said stimuli are recorded by the person on the 0 to 10 scale by using the keypad or mouse. Alternatively, the person can verbally communicate the level of distress to a person that records the indicated level or manually write it down themselves.

While not required for the present computerize test for HEV, a baseline test may be given to help the person become familiar with how to use the computer and scoring in which the person is asked to focus on a dot, which in the actual test that was given, was red while a friendly female face is shown on the left side of the screen for 0.4 seconds, after a one second mask. Thereafter, the person was asked to rate his or her level of distress on a 0 to 10 scale on a computer touch screen. The response is captured by the computer program and sent to a text file in which all of the person's responses are recorded. This preliminary baseline test was designed to acquaint the person with the method of scoring the emotional response to the image.

Next, a photograph of an angry man, symmetrical around the midline, was shown on the right side of the screen while the person was asked to focus on the red dot. The stressful image can be presented supraliminally, for more than 0.5 second. Similarly, if the image is presented with a mask and for short duration, it can be presented subliminally. For example, an image can be presented ten (10) times for 0.24 seconds, each time preceded and followed by a one (1.0) second mask. The person was then asked to rate his or her level of distress as before. As employed herein the term “mask” is a rapid presentation of a random pattern such as a repeating M. C. Escher image which is flashed on the screen for approximately one half (0.50) second to one (1.0) second before and after the stress image, which is presented for less than 20 milliseconds in order to facilitate the image's becoming subliminal or subconscious in the mind of the person. Notwithstanding the foregoing, the mask may be presented from 1/100^{th} of a second to approximately 2.0 seconds and the stimuli presented from 20 milliseconds to five (5) seconds. The test can have a duration anywhere from ten (10) seconds to one hour, with ten (10) minutes being often employed. It will be noted that the dot that is presented to the person was red and was positioned horizontally along the centerline of the screen, any color may be used or alternatively, a plurality of dots of differing or identical colors may be employed. The location of the dot (or other focal point such as in the figures) should be positioned so that when viewed the picture is in the person's lateral visual field.

In another aspect of the present invention a second stimulus is displayed on the screen after the first series described above. The second stimulus comprises at least one focal point displayed on the screen for the person to fixate on and as in the above first series; the focal point is positioned so that the images are seen in the person's other (left or right) lateral visual field. The second image on one side of the screen spaced away from said focal point, and the second stimulus presented to the person on the screen for a preselected time preceded and followed by a mask for a preselected time. The second stimulus is repeated a preselected number of times. The respective second focal point and said second image are presented on opposite sides of the screen from the respective first stimulus and first focal point. Recording the person's responses to the second stimulus is accomplished as above. The parameters of the focal point, stimulus and mask are essentially the same as those of the first stimulus. The entire sequence of trials was then repeated. The HEV score was computed as the difference between the left-sided trials minus the right-side trials. The HEV so computed meant that the positive number represented more distress to the left sided photograph which would suggest that the right hemisphere had a more negative valence and appropriate treatment actions can be taken based on the conclusions reached from the acquired data. Additionally, the test can be employed to determine the efficacy of a treatment. For example, after a light treatment to the positive hemisphere the patient's negative reaction to the computer test should be lessened. The test can also be used as a treatment, for example, if pictures of a pleasant loving person are shown to the troubled hemisphere.

The test of the present invention is presented to the person using a computer which displays a series of preselected stimuli. The stimuli are displayed on the computer screen and comprise a picture and a focal point for the person to fixate on. The focal point is positioned on the screen spaced apart from the picture so that picture on the extreme side of the screen is seen in the person's lateral visual field when the person fixates on the focal point. The focal point and the picture are displayed to the person for a preselected period of time to induce stress or comfort as the mental state associated with one of the cerebral hemispheres by stimulating the contralateral hemisphere. The test may be varied and modified according to the foregoing apparatus description.

The foregoing embodiments and examples are to be considered illustrative, rather than restrictive of the invention, and those modifications which come within the meaning and range of equivalence of the claims arte to be included therein.

That which is claimed is:

1. An apparatus for determining the hemispheric emotional valance of a person to assist in the selection of appropriate treatment modalities for cognitive enhancement, emotional well-being, psychiatric disorders including anxiety, depression, PTSD, drug abuse; neurological disorders including Alzheimer's disease, Parkinson's disease and traumatic brain injury, comprising:
a computer adapted to output a series of preselected stimuli,
a screen adapted to display said preselected stimuli to the person and wherein said preselected stimuli comprise:
a picture; and
a focal point for the person to fixate on and further, wherein the focal point is positioned on the screen spaced apart from the picture so that picture on the extreme side of the screen is seen in the person's lateral visual field when the person fixates on the focal point;
wherein said focal point and said picture are displayed to the person for a preselected period of time to induce stress or comfort as the mental state associated with one of the cerebral hemispheres by stimulating the contralateral hemisphere.

2. The apparatus according to claim 1 wherein the stimuli are presented for between 10 seconds to one hour.

3. The apparatus according to claim 1 wherein said focal point comprises a plurality of spaced apart points.

4. The apparatus according to claim 3 wherein said spaced apart points comprise dots and wherein each dot is unique thereby allowing the subject to choose which focal point is most effective in eliciting a psychological response.

5. The apparatus according to claim 4 wherein each of said dots are of a different color.

6. The apparatus according to claim 1 wherein said stimuli are in horizontal alignment along the horizontal centerline of said screen.

7. The apparatus according to claim 1 wherein said focal point is positioned medially of the picture for the person to fixate on such that the person sees the picture in their lateral visual field; said focal point and said picture being displayed to the person for a preselected period of time to induce stress or comfort the mental state associated with one of the cerebral hemispheres by stimulating the contralateral hemisphere.

8. The apparatus according to claim 7 wherein said picture is selected from the group consisting of an upsetting, neutral or pleasant.

9. The apparatus according to claim 1 further including an input device adapted to communicate with said computer for the person to input information relating to their response to the stimuli and wherein said computer is constructed and arranged to record and store the input.

10. An apparatus for determining the hemispheric emotional valence of a person to assist in the selection of appropriate treatment modalities for cognitive enhancement, emotional well-being, psychiatric disorders including anxiety, depression, PTSD, drug abuse; neurological disorders including Alzheimer's disease, Parkinson's disease and traumatic brain injury, comprising:
a computer adapted to output a series of preselected stimuli,
a screen adapted to display said preselected stimuli to the person and wherein said preselected stimuli comprise;
a baseline picture and focal point and further, wherein the baseline stimulus displayed on said screen comprises at least one focal point for the person to fixate on and further, wherein the focal point is positioned on the screen so that the baseline picture is seen in the person's lateral visual field;
the baseline picture being positioned on one side of the screen spaced away from and being presented simultaneously with said focal point, the baseline stimulus presented to the person for a first preselected time and being repeated a preselected number of times;
a first stimulus displayed on said screen and wherein said first stimulus comprises at least one focal point for the person to fixate on and a first picture, wherein the focal point is positioned so that the picture is seen in the person's lateral visual field; and
the first picture being located on one side of the screen spaced away from and presented with said focal point, said first stimulus presented to the person for a first preselected time, said first stimulus being repeated a preselected number of times;
a second stimulus displayed on said screen and wherein said second stimulus comprises at least one focal point for the person to fixate on and a second picture and further, wherein the focal point is positioned so that the second picture is seen in the person's lateral visual field; and
the second picture being positioned on one side of the screen spaced away from said focal point, said second stimulus presented to the person on the screen for a preselected time, said second stimulus being repeated a preselected number of times and further; wherein the respective second focal point and said second picture are on opposite sides of the screen from the respective first stimulus and first focal point.

11. The apparatus according to claim 10 further including an input device adapted to communicate with said computer and to transmit the person's responses to said respective stimuli to the computer for archiving.

12. The apparatus according to claim 10 wherein the stimuli are presented for between 10 seconds to one hour.

13. The apparatus according to claim 12 wherein the stimuli are presented for between 20 milliseconds and 5.0 seconds.

14. The apparatus according to claim 10 further including a mask image presented to the person on said screen and wherein the mask signal appears between each image in each series of the respective first and second stimuli and wherein said mask image is presented to one lateral visual field after which the patient scores his response to the stimuli images, then stimuli image is presented to the opposite visual field after which the patent scores his responses to the respective stimuli images;
whereby the clinician can determine if stimuli images presented to one lateral visual field versus the other get a greater subjective response from the patient.

15. A method for determining the hemispheric emotional valance of a person to assist in the selection of appropriate treatment modalities for cognitive enhancement, emotional well-being, psychiatric disorders including anxiety, depression, PTSD, drug abuse; neurological disorders including Alzheimer's disease, Parkinson's disease and traumatic brain injury, comprising:
presenting to the person using a computer a series of preselected stimuli,
displaying on a screen the preselected stimuli to the person and wherein the preselected stimuli comprise:
a picture and a focal point presented on the screen for the person to fixate on and further, wherein the focal point is positioned on the screen spaced apart from the picture so that picture on the extreme side of the screen is seen in the person's lateral visual field when the person fixates on the focal point;
wherein the focal point and said picture are displayed to the person for a preselected period of time to induce stress or comfort as the mental state associated with one of the cerebral hemispheres by stimulating the contralateral hemisphere.

16. The method according to claim 15 wherein the focal point comprises a plurality of spaced apart points.

17. The method according to claim 16 wherein the spaced apart points comprise dots and wherein each dot is unique.

18. The method according to claim 17 wherein the dots are each of a different color.

19. The method according to claim 15 wherein the stimuli are in horizontal alignment along the horizontal centerline of the screen.

20. The method according to claim 15 wherein the stimuli further include:
presenting a focal point on the screen and wherein the focal point is positioned on the lateral portion of the screen for the person to fixate on such that the person sees the image in their lateral visual field;
a first picture; and
wherein the focal point and the picture are displayed to the person for a preselected period of time to induce stress or comfort the mental state associated with one of the cerebral hemispheres by stimulating the contralateral hemisphere.

21. The method according to claim 20 wherein the picture is selected from the group consisting of an upsetting, neutral or pleasant.

22. The method according to claim 15 further including an input device adapted to communicate with the computer for the person to input information relating to their response to the stimuli and wherein the computer is constructed and arranged to record and store the input.

23. The method according to claim 15 wherein the stimuli are presented for between 10 seconds to 10 minutes
